(19) United States
(12) Patent Application Publication
(10) Pub. No.: US 2021/0311018 A1
(43) Pub. Date: Oct. 7, 2021

(54) CLOUD-BASED PORTABLE SYSTEM FOR NON-INVASIVE REAL-TIME URINALYSIS

(71) Applicant: Analog Devices, Inc., Wilmington, MA (US)

(72) Inventors: J. Brian HARRINGTON, Revere, MA (US); Hari CHAUHAN, Lexington, MA (US); Teoman Emre USTUN, Lexington, MA (US); Alexander GRAY, Andover, MA (US)

(73) Assignee: Analog Devices, Inc., Wilmington, MA (US)

(21) Appl. No.: 17/347,062

(22) Filed: Jun. 14, 2021

Related U.S. Application Data
(63) Continuation of application No. PCT/US2019/064146, filed on Dec. 3, 2019.
(60) Provisional application No. 62/781,284, filed on Dec. 18, 2018.

(51) Int. Cl. G01N 33/493; G01N 33/487; G01N 21/29; G01N 21/01

(52) U.S. Cl. CPC G01N 33/493; G01N 33/48792; G01N 2021/0137; G01N 21/01; G01N 21/29

(57) ABSTRACT

A method for implementing a cloud-based portable miniaturized system for performing non-invasive urinalysis in real time, the method comprising using an optical source to emit optical radiations at certain wavelengths through fluid in a fluid sampling medium; receiving the emitted optical transmissions at a photodetector; converting the received optical transmissions to digital data; accumulating the digital data for a first time period; and periodically transmitting the accumulated digital data to a cloud service for further processing.

### CROSS-REFERENCE TO RELATED APPLICATIONS

This application claims the benefit of and priority to U.S. Patent Application Ser. No. 62/781,284, filed Dec. 18, 2018, entitled “CLOUD-BASED PORTABLE SYSTEM FOR NON-INVASIVE REAL-TIME URINALYSIS,” and PCT Patent Application No. PCT/US2019/064146, filed Dec. 3, 2019, entitled “CLOUD-BASED PORTABLE SYSTEM FOR NON-INVASIVE REAL-TIME URINALYSIS”, each of which is incorporated herein by reference in its entirety.

### FIELD OF THE DISCLOSURE

This disclosure relates generally to the field of urinalysis systems and, more particularly, to a cloud-based portable miniaturized system for performing non-invasive urinalysis in real time.

### BACKGROUND

A urine analysis, or “urinalysis,” refers to a set of physical, chemical, and/or microscopic tests designed to detect and/or measure a variety of substances in the urine. Such substances may include byproducts of normal and abnormal metabolism, cells, cellular fragments, drugs and metabolites thereof, and bacteria, for example.

### SUMMARY OF THE DISCLOSURE

A method for implementing a cloud-based portable miniaturized system for performing non-invasive urinalysis in real time, the method comprising using an optical source to emit optical radiations at certain wavelengths through fluid in a fluid sampling medium; receiving the emitted optical transmissions at a photodetector; converting the received optical transmissions to digital data; accumulating the digital data for a first time period; and periodically transmitting the accumulated digital data to a cloud service for further processing.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIGS. 1A-1F illustrate a cloud-based portable miniaturized system for non-invasive real-time urinalysis in accordance with embodiments described herein;

FIG. 2 is a schematic block diagram of a cloud-based portable miniaturized system for non-invasive real-time urinalysis in accordance with embodiments described herein;

FIGS. 3A and 3B are flowcharts illustrating operation of a cloud-based portable miniaturized system for non-invasive real-time urinalysis in accordance with embodiments described herein; and

FIGS. 4A-4C are graphs illustrating the respective IR spectra of selected ones of parameters of interest; namely, urea, glucose, and uric acid, that may be detected using a cloud-based portable miniaturized system for non-invasive real-time urinalysis in accordance with embodiments described herein.

### DETAILED DESCRIPTION OF EXAMPLE EMBODIMENTS

Embodiments described herein comprise a cloud-based system for performing real time non-invasive urinalysis. Certain embodiments may be used to estimate the key parameters/biomarkers and/or test for the presence of drugs/drug metabolites (such as cocaine and THC and their metabolites), certain hormones (e.g., pregnancy hormones), and bacteria in a urine sample as set forth in Table 1 below:

| | | |
|---|---|---|
| Osmolality | Chloride | Nitrites* |
| Sodium | Urea | Blood* (RBC) |
| Uric acid | Phosphate | Urobilliruben* |
| Calcium | Magnesium | Ketones* |
| Glucose* | Total Protein* | Bilirubin* |
| Amylase | Potassium | Leukocytes* |
| Creatinine | Melb-Random | Specific Gravity* |
| THC and THC | Drugs and Drug | Bacteria and |
| Metabolites | Metabolites | Bacteria Toxins |
| Pregnancy Hormones | Cocaine and Cocaine | |
| | Metabolites | |

*Test parameters commonly found on urine test strips

### Table 1

Embodiments may also be extended for other applications, such as pregnancy tests, drug tests, etc. Additionally, embodiments may be extended to cover detection of bacteria in urine, either by detecting the bacteria itself or by detecting toxins produced by the bacteria as a signature thereof. Some embodiments are implemented as a spectroscopic method for collecting spectral information from urine samples and use cloud-based computational algorithms for determining the concentration levels of various parameters, such as those listed in Table 1 above. Embodiments may further be implemented as an attachment to a urine catheter tube or as an accessory in a washroom, where it may be attached to or separate from a urinal. Currently, spectroscopic methods used in hospital labs and similar environments are deployed using desktop spectrometers, require urine samples, and cannot be used to perform real-time analysis. For example, failing to perform a urinalysis on a patient in an intensive care unit (“ICU”) in a timely manner may prove life threatening.

Embodiments described herein comprise a complete cloud-connected system that includes a system housing, a spectrometric system, a temperature sensor, and an electronic system. In certain embodiments, the spectrometric system includes a source, which may be implemented using a Quantum Cascade Laser (“QCL”), and a detector. In some embodiments, the QCL functionality may be replaced by an ADSC100, available from Analog Devices of Norwood, Mass., which is a miniaturized near infrared (“NIR”) spectrometer. The ADSC100 contains a broadband NIR light source, optical filters and detectors comprising a miniaturized NIR spectrometer. The QCL functionality may also be replaced by a plurality of discrete LEDs. The detector 110 may be implemented using, for example, any one or more of SCiO detectors, combinations of LEDs/quantum dots (“QDs”), and/or photodetectors for discrete wavelengths not limited by numbers of LEDs/QDs plus photodetectors.

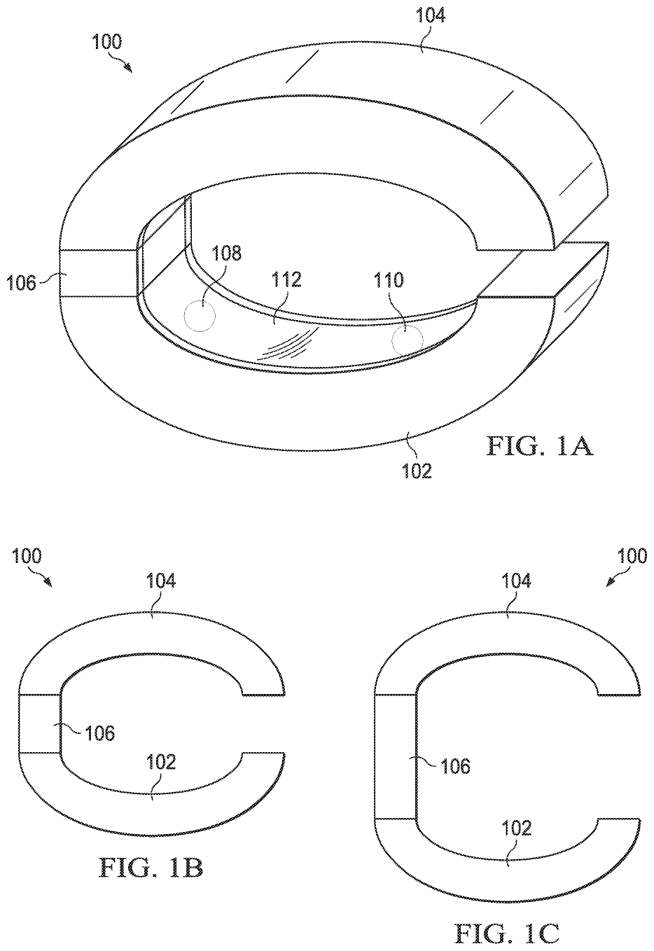

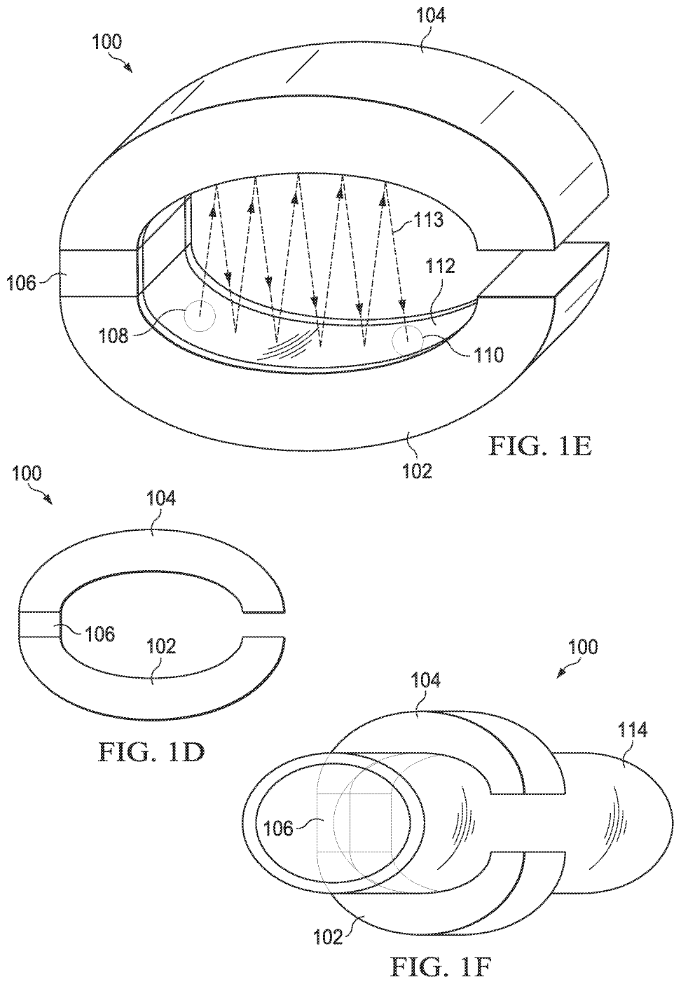

FIG. 1A is a perspective illustration of a cloud-based portable handheld system 100 for non-invasive real-time urinalysis in accordance with embodiments described herein. As shown in FIG. 1A, the system 100 includes a system housing including a base arm 102 and a reflective arm 104 connected to one another via an adjustment, or adjustable, arm 106, which allows easy fixture of the system 100 around tubes or beakers having a variety of shapes, such as illustrated in FIGS. 1B-1D. The arms 102, 104, may be constructed of any appropriate material and may be flexible as needed. The system 100 may be designed to slide over an end of a tube or beaker or may have a hinge such that the arms 102, 104, open to accept the tube or beaker and then close to clamp around the tube/beaker. The system 100 also includes a source 108 and a detector 110 (e.g., an infrared (“IR”) detector) disposed opposite one another on the inside of system housing (i.e., arms 102-106). The inside surfaces of arms 102-106 are coated with a reflective material 112 to permit radiation to travel from the source 108 to the detector 110 via the urine sample contained in the tube encircled by arms 102-106. Reflective coating 112 and the configurable detector position also enables adjustment of path length for better sensitivity. As an example, the detector position with respect to the source (QCL, or NIR/IR) along the optical path provides flexibility to increase or decrease the optical path length as needed by increasing/decreasing the number of transmissions/reflections between the transmitter and receiver. FIG. 1E illustrates a radiation path 113 from the source 108 to the detector 110, as aided by reflective coating 112. FIG. 1F illustrates the system 100 in use with a catheter tube section 114, for example, in which the arms 102-106 encircle the tube section.

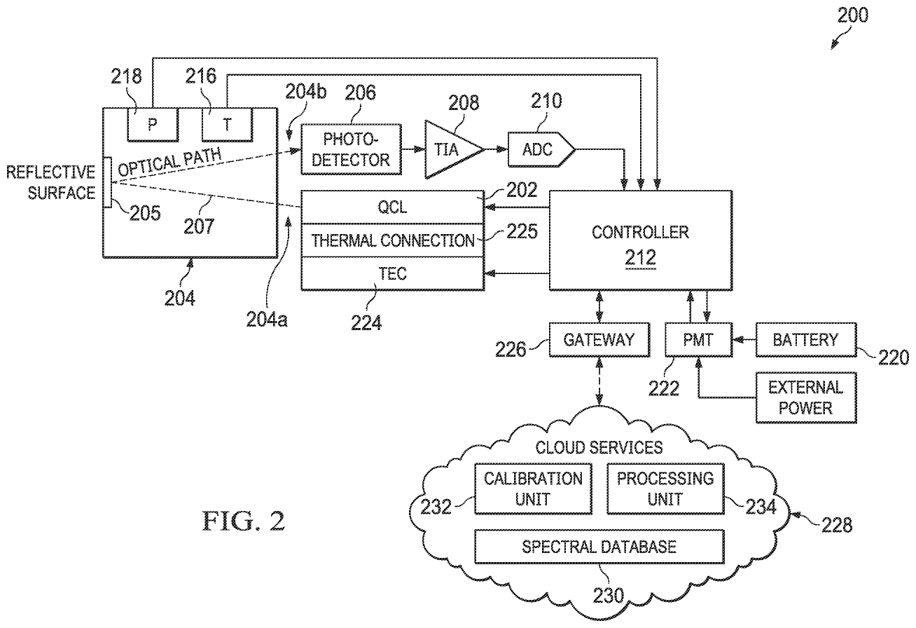

The system housing is a complete system comprising the two arc-shaped arms 102, 104, which are connected to one another via adjustable arm 106, which allows easy size adjustment of the system to fit accommodate different urine sampling media, which may include a urine catheter tube and a small glass beaker. In the embodiment illustrated in FIG. 1A, both the source 108 and the detector 110 may be disposed on the base arm 102 and/or the reflective arm 104. As previously noted, in one embodiment, as will be described in greater detail below, the source 108 is implemented using a QCL (and/or miniaturized NIR/IR spectrometer such as the aforementioned ADSC100, or discrete LEDs and detector combinations), which emits radiation having a predetermined wavelength. The inner surfaces of the reflective arm 104 and the base arm 102 are coated with IR reflective material or IR reflective mirrors to allow the emitted radiation to experience multiple reflections through the urine sample before actually reaching the detector. In certain embodiments, the system housing is equipped with a temperature sensor to measure temperature of the urine sample. Alternatively, the IR radiation can also be used to measure the temperature of the urine sample. The system 100 is also equipped with the required electronic system, shown in FIG. 2, to convert the received light into digital data, calculate the raw spectral power density (spectrum) and to transmit the measured temperature and the spectrum of the urine sample to the cloud.

Referring now to FIG. 2, illustrated therein is a system block diagram of a spectrometer system 200 for use in implementing a cloud-based portable handheld system, such as system 100, for non-invasive real-time urinalysis in accordance with embodiments described herein. In the illustrated embodiment, the system 200 comprises a chip-scale QCL-based spectrometer system. The spectrometer system 200 operates by measuring the optical transmission of the laser beam through the breath/gas captured in the breath chamber. As shown in FIG. 2, the spectrometer system 200 includes a chip-scale QCL source 202 that transmits highly focused optical radiations at certain wavelengths. The radiated wavelengths can be adjusted by changing the operating temperature of the QCL source 202 providing the optical radiations in the near and mid infrared region of the electromagnetic spectrum, effectively covering the range of 0.7 μm to 20 μm. In addition, the power of the radiation emitted by the QCL source 202 may be tuned by tuning the operating conditions of the source. Optical radiation emitted by the QCL source 202 enters a chamber 204 from a first end 204a, experiences multiple reflections inside the chamber (e.g., at a reflective surface 205) while traversing therethrough, and is received by a photodetector 206 located at a second end 204b of the chamber opposite the first end 204a. An optical path of the of the optical radiation through the chamber 204 is represented in FIG. 2 by an arrow 207. The optical radiation, or light, incident on the photodetector 206 generates a current, which is amplified and converted to a voltage by a transimpedance amplifier (“TIA”) stage 208. The voltage is then digitized by an analog-to-digital converter (“ADC”) 210 and the resulting digital data is processed by a controller 212. It will be noted that, as represented in FIG. 2, multiple photodetectors, each with a corresponding TIA stage, may be deployed as deemed advantageous for implementing various embodiments.

The spectrometer system 200 must be able to measure the optical transmission of the urine sample over a range of frequencies sufficient to uniquely determine the concentration of selected biomarkers, such as urea, creatinine, osmolality, etc., as presented in Table 1. The construction details of the QCL source 202 are chosen in order meet the frequency range requirements. The QCL 202 is constructed with a series of quantum wells. The physical size of the wells determines the nominal frequency of the emitted light, with each well enabling a narrow frequency band of light to be transmitted. The well that is activated can be controlled, thereby enabling the output frequency of the QCL 202 to be selected. Additionally, the frequency of the emitted light varies with temperature; accurately varying the temperature of the QCL enables the frequency to be continuously swept across different frequencies ranging from NIR to mid infrared (“MIR”) range. The combination of the nominal frequencies selected and the frequency sweeps allow enough of the frequency band to be scanned to measure the concentration of the various biomarkers.

On the optical receiver side, the photodetector 206 and optical filter combination has a relatively uniform bandwidth over the transmitted light frequencies, with any variations therein being removed using a calibration routine. As a result, the photodetector 206 has minimal impact on the overall frequency transfer of the system 200. To reduce the power consumption of the laser 202 and remove the low frequency noise of the TIA and the ADC, a synchronous demodulation technique is used for the optical signal measurement. This technique involves pulsing the laser 202 and synchronously sampling the response at the output of the TIA 208. The pulse width of the laser 202 is selected based on the settling time requirements of the TIA 208 stage, with a typical pulse width being approximately 1 μs. The pulse, or modulation, rate of the laser 202 involves a tradeoff between the 1/f frequency of the TIA 208 and the sample rate of the ADC 210. The modulation rate of the laser 202 should be above the 1/f frequency to reduce the impact of the electrical noise, but no so high as to require a higher power, costlier ADC and impose additional processing burden on the controller 212. A typical modulation frequency may be approximately 10 KHz. The ADC 210 will normally sample the waveform at 4 times the modulation frequency to use IQ sampling, which improves the accuracy of the measurement.

In certain embodiments, auxiliary sensors can be used to improve the calibration of the measurement. For example, a temperature sensor 216 and a pressure sensor 218, and relative humidity may be optionally employed inside the chamber 204 to measure air temperature and pressure within the chamber. The temperature and pressure can be used separately or in tandem in the calibration routine performed in the cloud. Because the system 200 is designed to be portable, it includes a battery 220 and power management functionality (“PMT”) 222 as well.

In general, the functions of the controller 212 include synchronously triggering the light source and the ADC sampling, accumulating and compressing the ADC data, operating a thermoelectric cooler (“TEC”) 224 to maintain the desired temperature, and communicating with a gateway 226 to transmit data and instructions to and from cloud services 228. The PMT 222 provides the required supply voltages for the electronics from the battery 220 or an externally supplied power source. The PMT 222 also recharges the battery from an externally supplied power source. The QCL 202 is a multi-wavelength laser that is excited from a high energy LED. The QCL down converts LED optical energy into an array of longer wavelengths, which are selected to align with the absorption wavelengths of the biomarkers under test.

The TEC 224 is provided to stabilize the temperature of the QCL 202 (via a thermal connection 225) as necessary to calibrate and stabilize the QCL operation. The gateway 226 provides a communications link between the controller 212 and the cloud services 228. In one embodiment, the communications link includes a wireless connection, such as Bluetooth low energy (“BLE”), WIFI, or LTE Cat-M.

The cloud services infrastructure includes several elements, including a spectral database 230, a calibration unit 232, and a processing unit 234, which includes preprocessing algorithms, chemometric models, and lookup tables. In one embodiment, the spectral database is built using the described system and urine samples with known concentrations of various biomarkers at various humidity and temperature conditions. It consists of optical transmission measurements of the biomarkers at the wavelengths of interest in near and mid infrared supported by the chip-scale QCL.

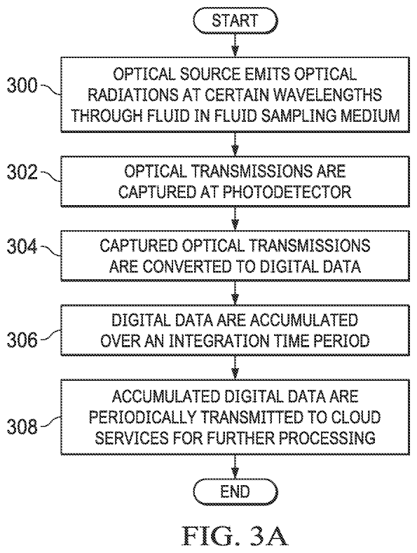

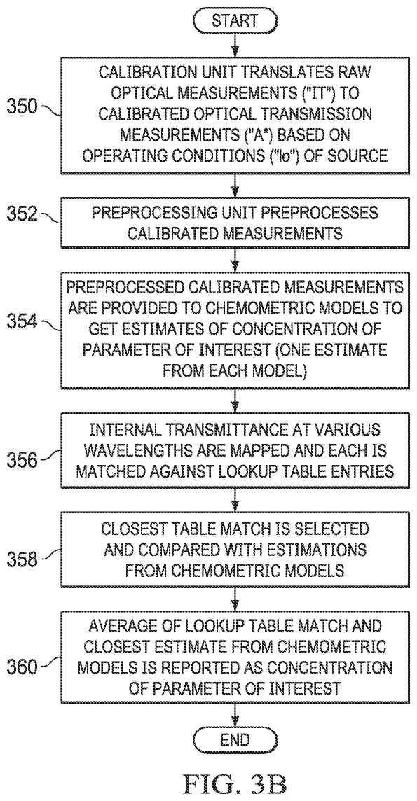

FIGS. 3A and 3B are flowcharts illustrating operation of a cloud-based portable miniaturized system for non-invasive real-time urinalysis in accordance with embodiments described herein. Referring to FIG. 3A, in step 300, an optical source, such as a QCL, emits a laser beam comprising highly focused optical radiations at certain wavelengths through fluid (e.g., urine) contained in a fluid sampling medium. In step 302, the resulting optical transmissions are captured at a photodetector and converted into digital data in step 304. In step 306, the digital data are accumulated over an integration time period and in step 308, the accumulated data are periodically transmitted to cloud services for further processing.

Referring now to FIG. 3B, in step 350, the calibration unit translates the raw optical measurements captured by the photodetector (“IT”) to a calibrated optical transmission measurement (“A”) based on the operating conditions (“Io”) of the chip-scale QCL. The calibration unit basically generates the optical transmission based on the Beer-Lambert-Bouguer law defined as:

A=−log 10(IT/Io)

where:
IT is the monochromatic radiant power transmitted by the absorbing medium;
Io is the monochromatic radiant power incident on the medium; and
τi is the internal transmittance (=IT/Io).

The processing unit consists of several processing blocks, such as preprocessing algorithms, chemometric models, and lookup tables. In step 352, preprocessing algorithms preprocess the calibrated transmission (i.e., the output of the calibration unit) and support various elements, such as log 10, In, first and second derivatives, averaging, Standard Normal Variate (“SNV”), autoscaling, baseline correction, and Multiplicative Scatter Correction (“MSC”), for example. Chemometric models include at least three such models based on Multiple Linear Regression (“MLR”) and Principal Components Regression (“PCR”), and Partial Least Square (“PLS”) regression. Lookup tables include ratios of internal transmittance (“τi”) at various wavelengths for different concentrations of the biomarkers. The look-up table is constructed along with the database using the samples of biomarkers in urine samples under various operating conditions such as humidity, temperature, and QCL source power (“Io”) etc. Look-up tables are utilized as a mean to validate the estimates made by the chemometric models.

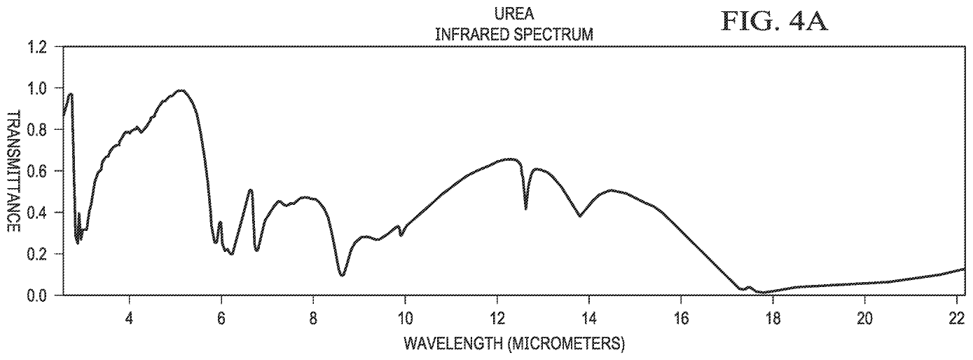

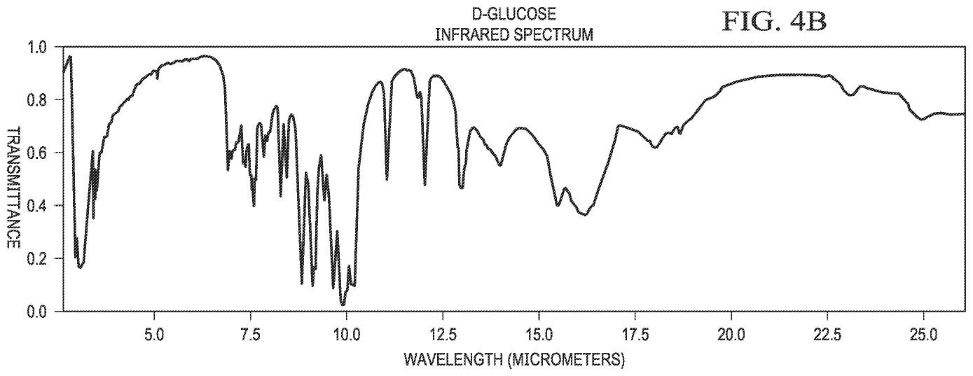

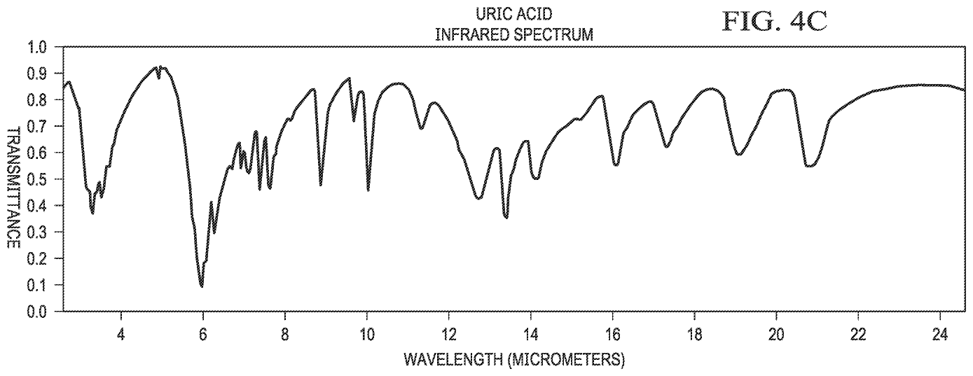

As previously noted, the raw optical measurements (“IT”) measured at the photodetector are calibrated by the calibration unit to generate the internal transmittance (“τi”) at the various supported NIR and MIR wavelengths. These calibrated measurements are preprocessed and in step 354, the measurements are provided to the three chemometric models to estimate the concentration of the parameter of interest, with each model providing one estimate. The preprocessing scheme, comprising a combination of preprocessing algorithms, is fixed for a given chemometric model. In addition, in step 356, the internal transmittance (“τi”) measured at various wavelengths is mapped and matched against lookup table entries. In step 358, the estimated concentration that provide the closest match to the lookup table entry is then picked and compared to the estimations from the chemometric model. The average of look-up table match and the closest estimate of it from the chemometric models is then reported back to the user as the measured parameter's concentration in step 360. Table 2 lists the reference values of the parameters listed in Table 1. FIGS. 4A-4C are graphs illustrating the IR spectrum of selected ones of the parameters listed in Table 1; namely, urea, D-glucose, and uric acid. The described system targets the spectral signatures of various biomarkers available in the region of 0.7 μm-10 μm.

TABLE 2

| Parameter | Random Sample | 24-Hour Sample | |
|---|---|---|---|
| Osmolality | 38-1400 mOsm/kg | NA | |
| Sodium | 20 mEq/L | 100-260 | mmol/24 h |
| Potassium | NA | 25-100 | mmol/24 h |
| Urea | NA | 12-20 | g |
| Uric Acid | NA | 250-750 | mg/24 h |
| Total Protein | NA | <100 | mg/24 h |
| Glucose | 0 or trace | 0 or trace | |
| Albumin | 0 or trace | <30 | mg/24 hour |
| Creatinine | NA | 15-25 | mg/kg/24 h |

Weight based measurement,

| | |
|---|---|
| Bilirubin | 0 or trace |

| | | | |
|---|---|---|---|
| Urobilinogen | NA | 0.05-2.5 | mg/24 h |
| Chloride | NA | 80-250 | mmol/day |
| Calcium | NA | 100-300 | mg/day |
| Magnesium | NA | 51-269 | mg/24 hr |

| | | |
|---|---|---|
| Phosphate | NA | 79-94% of filtered load |
| | | Not a specified amount |

| | |
|---|---|
| RBC | 0 or trace |
| Leukocytes | 0 or trace |

Embodiments described herein are a cloud-based portable handheld system for non-invasive real-time urinalysis in which a tunable chip-scale QCL laser is used and emits different wavelengths by changing the temperature. Additionally, the fact that the system is small, handheld, and easily portable allows easy fixture of the system to the sampling medium. Still further, the system is cloud-based and provides algorithms for data processing offering real time urinalysis.

In Example 1, a method for implementing a cloud-based portable miniaturized system for performing non-invasive urinalysis in real time may comprise translating raw optical measurement (“IT”) to calibrated optical transmission measurements (“A”) based on operating conditions (“Io”) of an optical transmission source of the system; preprocessing the calibrated optical transmission measurements; providing the preprocessed calibrated optical transmission measurements to at least one chemometric model to obtain an estimate of a concentration of a parameter of interest; mapping internal transmittance at a first wavelength and matching the mapped internal transmittance to a lookup table entry; and reporting an average of the matched lookup table entry and the estimate from the at least one chemometric model as a concentration of the parameter of interest.

In Example 2, the method of Example 1 may further include the translation being performed in accordance with A=−log 10(IT/Io).

In Example 3, the methods of any of Examples 1-2 may further include the parameter of interest comprising at least one of osmolality, sodium, potassium, urea, uric acid, total protein, glucose, albumin, creatinine, bilirubin, urobilinogen, chloride, calcium, magnesium, phosphate, RBC, and leukocytes.

In Example 4, the methods of any of Examples 1-3 may further include the parameter of interest comprising at least one of pregnancy hormone, THC, THC metabolites, cocaine, cocaine metabolites, bacteria, and toxins produced by bacteria.

In Example 5, a method for implementing a cloud-based portable miniaturized system for performing non-invasive urinalysis in real time may comprise using an optical source to emit optical radiations at certain wavelengths through fluid in a fluid sampling medium; receiving the emitted optical transmissions at a photodetector; converting the received optical transmissions to digital data; accumulating the digital data for a first time period; and periodically transmitting the accumulated digital data to a cloud service for further processing.

In Example 6, the method of Example 5 may further include the optical source comprising a Quantum Cascade Laser (“QCL”).

In Example 7, the method of any of Examples 5-6 may further include the optical source comprising at least one of a miniaturized near infrared (“NIR”) spectrometer and at least one discrete LED, at least one quantum dot (“OD”), and an SCiO sensor.

In Example 8, the method of any of Examples 5-7 may further include the photodetector comprising at least one of at least one discrete LED, at least one quantum dot (“OD”), and an SCiO sensor.

In Example 9, the method of any of examples 5-7 may further include adjusting an optical path length between the source and the detector by adjusting a number of reflections experienced by the optical radiations.

In Example 10, an apparatus for implementing a cloud-based portable miniaturized system for performing non-invasive urinalysis in real time may include a system housing configured to encircle a urine collection medium; an optical source disposed at a first side of the system housing; and an optical detector disposed at a second side of the system housing opposite the first side thereof; wherein radiation emitted from the source travels through fluid disposed within the urine collection medium and is detected by the detector.

In Example 11, the apparatus of Example 10 may further include the system housing including a first curved arm having a first end and a second end; a second curved arm having a first end and a second end; and an adjustment arm connected between the first end of the first curved arm to the first end of the second curved arm such that a space exists between the second end of the first curved arm and the second end of the second curved arm.

In Example 12, the apparatus of any of Examples 10-11 may further include a reflective coating disposed on an inside of the system housing to adjust an optical path of the radiation through the fluid disposed within the urine collection medium.

In Example 13, the apparatus of any of Examples 10-12 may further include the optical source comprising at least one of a Quantum Cascade Laser (“QCL”), a plurality of discrete LEDs and a miniaturized near infrared (“NIR”) spectrometer.

In Example 14, the apparatus of any of Examples 10-13 may further include the optical detector comprising at least one of at least one discrete LED, at least one quantum dot (“OD”), and an SCiO sensor.

In Example 15, the apparatus of any of Examples 10-14 may further include a sensor for measuring a temperature of the fluid disposed within the urine collection medium.

In Example 16, the apparatus of any of Examples 10-15 may further include electronics for converting the detected radiation into digital data.

In Example 17, the apparatus of any of Examples 10-16 may further include electronics for calculating a raw spectral power density of the detected radiation.

In Example 18, the apparatus of any of Examples 10-17 may further include a gateway device for transmitting the digital data and the raw spectral power density to a cloud service for processing.

In Example 19, the apparatus of any of Examples 10-18 may further include the urine collection medium comprising a catheter tube.

In Example 20, the apparatus of any of Examples 10-19 may further include the urine collection medium comprising a glass receptacle.

In Example 21, the apparatus of any of Examples 10-20 may further include a battery and power management functionality.

In Example 22, the apparatus of any of Examples 10-21 may further include the optical source emitting highly focused optical radiations at selected wavelengths.

In Example 23, the apparatus of any of Examples 10-22 may further include the selected wavelengths being adjusted within a range of 0.7 μm to 20 μm by changing an operating temperature of the optical source.

In Example 24, the apparatus of any of Examples 10-23 may further include the optical detector comprising a photodetector and an optical filter having a relatively uniform bandwidth over transmitted light frequencies.

In Example 25, an apparatus for implementing a cloud-based portable miniaturized system for performing non-invasive urinalysis in real time may include a system housing configured to encircle a urine collection medium, the system housing comprising: a first curved arm having a first end and a second end; a second curved arm having a first end and a second end; and an adjustment arm connected between the first end of the first curved arm to the first end of the second curved arm such that a space exists between the second end of the first curved arm and the second end of the second curved arm. The apparatus may further include an optical source disposed at a first side of the system housing; and an optical detector disposed at a second side of the system housing opposite the first side thereof; wherein radiation emitted from the source travels through fluid disposed within the urine collection medium and is detected by the detector.

In Example 26, the apparatus of Example 25 may further include a reflective coating disposed on an inside of each of the first and second curved arms to adjust an optical path of the radiation through the fluid disposed within the urine collection medium.

In Example 27, the apparatus of any of Examples 25-26 may further include the optical source comprising a Quantum Cascade Laser (“QCL”).

In Example 28, the apparatus of any of Examples 25-27 may further include the optical source comprising at least one of a miniaturized near infrared (“NIR”) spectrometer and a plurality of discrete LEDs.

In Example 29, the apparatus of any of Examples 25-28 may further include the optical detector comprising at least one of at least one discrete LED, at least one quantum dot (“QD”), and an SCiO sensor.

In Example 30, the apparatus of any of Examples 25-29 may further include a sensor for measuring a temperature of the fluid disposed within the urine collection medium.

In Example 31, the apparatus of any of Examples 25-30 may further include electronics for at least one of converting the detected radiation into digital data and calculating a raw spectral power density of the detected radiation.

In Example 32, the apparatus of any of Examples 25-31 may further include a gateway device for transmitting the digital data and the raw spectral power density to a cloud service for processing.

In Example 33, the apparatus of any of Examples 25-32 may further include the urine collection medium comprising at least one of a catheter tube and a glass receptacle.

In Example 34, the apparatus of any of Examples 25-33 may further include a battery and power management functionality.

In Example 35, the apparatus of any of Examples 25-34 may further include the optical source emitting highly focused optical radiations at selected wavelengths.

In Example 36, the apparatus of any of Examples 25-35 may further include the selected wavelengths being adjusted within a range of 0.7 μm to 20 μm by changing an operating temperature of the optical source.

In Example 37, the apparatus of any of Examples 25-36 may further include a photodetector and an optical filter having a relatively uniform bandwidth over transmitted light frequencies.

It should be noted that all of the specifications, dimensions, and relationships outlined herein (e.g., the number of elements, operations, steps, etc.) have only been offered for purposes of example and teaching only. Such information may be varied considerably without departing from the spirit of the present disclosure, or the scope of the appended claims. The specifications apply only to one non-limiting example and, accordingly, they should be construed as such. In the foregoing description, exemplary embodiments have been described with reference to particular component arrangements. Various modifications and changes may be made to such embodiments without departing from the scope of the appended claims. The description and drawings are, accordingly, to be regarded in an illustrative rather than in a restrictive sense.

Note that with the numerous examples provided herein, interaction may be described in terms of two, three, four, or more electrical components. However, this has been done for purposes of clarity and example only. It should be appreciated that the system may be consolidated in any suitable manner. Along similar design alternatives, any of the illustrated components, modules, and elements of the FIGURES may be combined in various possible configurations, all of which are clearly within the broad scope of this Specification. In certain cases, it may be easier to describe one or more of the functionalities of a given set of flows by only referencing a limited number of electrical elements. It should be appreciated that the electrical circuits of the FIGURES and its teachings are readily scalable and may accommodate a large number of components, as well as more complicated/sophisticated arrangements and configurations. Accordingly, the examples provided should not limit the scope or inhibit the broad teachings of the electrical circuits as potentially applied to myriad other architectures.

It should also be noted that in this Specification, references to various features (e.g., elements, structures, modules, components, steps, operations, characteristics, etc.) included in “one embodiment”, “exemplary embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It should also be noted that the functions related to circuit architectures illustrate only some of the possible circuit architecture functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

Note that all optional features of the device and system described above may also be implemented with respect to the method or process described herein and specifics in the examples may be used anywhere in one or more embodiments.

The ‘means for’ in these instances (above) may include (but is not limited to) using any suitable component discussed herein, along with any suitable software, circuitry, hub, computer code, logic, algorithms, hardware, controller, interface, link, bus, communication pathway, etc.

Note that with the example provided above, as well as numerous other examples provided herein, interaction may be described in terms of two, three, or four network elements. However, this has been done for purposes of clarity and example only. In certain cases, it may be easier to describe one or more of the functionalities of a given set of flows by only referencing a limited number of network elements. It should be appreciated that topologies illustrated in and described with reference to the accompanying FIGURES (and their teachings) are readily scalable and may accommodate a large number of components, as well as more complicated/sophisticated arrangements and configurations. Accordingly, the examples provided should not limit the scope or inhibit the broad teachings of the illustrated topologies as potentially applied to myriad other architectures.

It is also important to note that the steps in the preceding flow diagrams illustrate only some of the possible signaling scenarios and patterns that may be executed by, or within, communication systems shown in the FIGURES. Some of these steps may be deleted or removed where appropriate, or these steps may be modified or changed considerably without departing from the scope of the present disclosure. In addition, a number of these operations have been described as being executed concurrently with, or in parallel to, one or more additional operations. However, the timing of these operations may be altered considerably. The preceding operational flows have been offered for purposes of example and discussion. Substantial flexibility is provided by communication systems shown in the FIGURES in that any suitable arrangements, chronologies, configurations, and timing mechanisms may be provided without departing from the teachings of the present disclosure.

Although the present disclosure has been described in detail with reference to particular arrangements and configurations, these example configurations and arrangements may be changed significantly without departing from the scope of the present disclosure. For example, although the present disclosure has been described with reference to particular communication exchanges, embodiments described herein may be applicable to other architectures.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke paragraph six (6) of 35 U.S.C. section 142 as it exists on the date of the filing hereof unless the words “means for” or “step for” are specifically used in the particular claims; and (b) does not intend, by any statement in the specification, to limit this disclosure in any way that is not otherwise reflected in the appended claims.

What is claimed is:

1. A method for implementing a cloud-based portable miniaturized system for performing non-invasive urinalysis in real time, the method comprising:
translating raw optical measurement (“IT”) to calibrated optical transmission measurements (“A”) based on operating conditions (“Io”) of an optical transmission source of the system;
providing the calibrated optical transmission measurements to at least one chemometric model to obtain an estimate of a concentration of a parameter of interest;
mapping internal transmittance at a first wavelength and matching the mapped internal transmittance to a lookup table entry; and
reporting an average of the matched lookup table entry and the estimate from the at least one chemometric model as a concentration of the parameter of interest.

2. The method of claim 1 further comprising preprocessing the calibrated optical transmission measurements, wherein the calibrated optical transmission measurements provided to the at least one chemometric model comprise the preprocessed calibrated optical transmission measurements.

3. The method of claim 1, wherein the translation is performed in accordance with A=−log 10(IT/Io).

4. The method of claim 1, wherein the parameter of interest comprises at least one of osmolality, sodium, potassium, urea, uric acid, total protein, glucose, albumin, creatinine, bilirubin, urobilinogen, chloride, calcium, magnesium, phosphate, RBC, and leukocytes.

5. The method of claim 1, wherein the parameter of interest comprises at least one of pregnancy hormone, THC, THC metabolites, cocaine, cocaine metabolites, bacteria, and toxins produced by bacteria.

6. A method for implementing a cloud-based portable miniaturized system for performing non-invasive urinalysis in real time, the method comprising:
using an optical source to emit optical radiations at certain wavelengths through fluid in a fluid sampling medium;
receiving the emitted optical transmissions at a photodetector;
converting the received optical transmissions to digital data;
accumulating the digital data for a first time period; and
periodically transmitting the accumulated digital data to a cloud service for further processing.

7. The method of claim 6, wherein the optical source comprises a Quantum Cascade Laser (“QCL”).

8. The method of claim 6, wherein the optical source comprises at least one of a miniaturized near infrared (“NIR”) spectrometer and at least one discrete LED, at least one quantum dot (“QD”), and an SCiO sensor.

9. The method of claim 6, wherein the photodetector comprises at least one of at least one discrete LED, at least one quantum dot (“QD”), and an SCiO sensor.

10. The method of claim 6 further comprising adjusting an optical path length between the source and the detector by adjusting a number of reflections experienced by the optical radiations.

11. Apparatus for implementing a cloud-based portable miniaturized system for performing non-invasive urinalysis in real time, the apparatus comprising:
a system housing configured to encircle a urine collection medium;
an optical source disposed at a first side of the system housing; and
an optical detector disposed at a second side of the system housing opposite the first side thereof;
wherein radiation emitted from the source travels through fluid disposed within the urine collection medium and is detected by the detector.

12. The apparatus of claim 11, wherein the system housing comprises:
a first curved arm having a first end and a second end;
a second curved arm having a first end and a second end; and
an adjustment arm connected between the first end of the first curved arm to the first end of the second curved arm such that a space exists between the second end of the first curved arm and the second end of the second curved arm.

13. The apparatus of claim 11 further comprising a reflective coating disposed on an inside of the system housing to adjust an optical path of the radiation through the fluid disposed within the urine collection medium.

14. The apparatus of claim 11, wherein the optical source comprises at least one of a Quantum Cascade Laser (“QCL”), a plurality of discrete LEDs and a miniaturized near infrared (“NIR”) spectrometer.

15. The apparatus of claim 11, wherein the optical detector comprises at least one of at least one discrete LED, at least one quantum dot (“QD”), and an SCiO sensor.

16. The apparatus of claim 11 further comprising a sensor for measuring a temperature of the fluid disposed within the urine collection medium.

17. The apparatus of claim 11 further comprising electronics for converting the detected radiation into digital data.

18. The apparatus of claim 17 further comprising electronics for calculating a raw spectral power density of the detected radiation.

19. The apparatus of claim 18 further comprising a gateway device for transmitting the digital data and the raw spectral power density to a cloud service for processing.

20. The apparatus of claim 11, wherein the urine collection medium comprises at least one of a catheter tube and a glass receptacle.
